(19) United States
(12) Patent Application Publication
(10) Pub. No.: US 2004/0186163 A1
(43) Pub. Date: Sep. 23, 2004

(54) Novel combination

(76) Inventors: Hilmar Bischoff, Wuppertal (DE); Johannes-Peter Stasch, Solingen (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/486,620

(22) PCT Filed: Aug. 5, 2002
(86) PCT No.: PCT/EP02/08701

(30) Foreign Application Priority Data
Aug. 17, 2001 (DE) 101 40 421.2

(51) Int. Cl. A61K 31/401; A61K 31/366; A61K 31/225

(52) U.S. Cl. 514/423; 514/460; 514/548

(57) ABSTRACT

A combination product which comprises as pharmaceutically active ingredient at least one active ingredient component A and at least one active ingredient component B is described, where active ingredient component A is a direct soluble guanylate cyclase stimulator and active ingredient component B is a lipid-lowering agent.

The two active ingredient components A and B can be administered either simultaneously or else sequentially, i.e. in the form of functional unit or separate from one another.

The present invention relates to a novel combination product comprising at least one lipid-lowering agent and at least one compound able to stimulate soluble guanylate cyclase.

One of the most important cellular transmission systems in mammalian cells is cyclic guanosine monophosphate (cGMP). Together with nitric oxide (NO), which is released from the endothelium and transmits hormonal and mechanical signals, it forms the NO/cGMP system. Guanylate cyclases catalyze the biosynthesis of cGMP from guanosine triposphate (GTP). The representatives of this family disclosed to date can be divided both according to structural features and according to the type of ligands into two groups: the particulate guanylate cyclases which can be stimulated by natriuretic peptides, and the soluble guanylate cyclases which can be stimulated by NO. The soluble guanylate cyclases consist of two subunits and very probably contain one heme per heterodimer, which is part of the regulatory site. The latter is of central importance for the mechanism of activation. NO is able to bind to the iron atom of heme and thus markedly increase the activity of the enzyme. CO is also able to attach to the central iron atom of heme, but the stimulation by CO is distinctly less than that by NO.

Through the production of cGMP and the regulation, resulting therefrom, of phosphodiesterases, ion channels and protein kinases, guanylate cyclase plays a crucial part in various physiological processes, in particular in the relaxation and proliferation of smooth muscle cells, in platelet aggregation and adhesion and in neuronal signal transmission, and in disorders caused by an impairment of the aforementioned processes.

Compounds, such as organic nitrates, whose effect is based on the release of NO have to date been exclusively used for the therapeutic stimulation of soluble guanylate cyclase. NO is produced by bioconversion and activates soluble guanylate cyclase by attaching to the central iron atom of heme. Besides the side effects, the development of tolerance is one of the crucial disadvantages of this mode of treatment.

Some substances which directly stimulate soluble guanylate cyclase, i.e. without previous release of NO, have been described in recent years, such as, for example, 3-(5′-hydroxymethyl-2′-furyl)-1-benzylindazole (YC-1, Wu et al., Blood 84 (1994), 4226; Mülsch et al., Br. J. Pharmacol. 120 (1997), 681), fatty acids (Goldberg et al, J. Biol. Chem. 252 (1977), 1279), diphenyliodonium hexafluorophosphate (Pettibone et al., Eur. J. Pharmacol. 116 (1985); 307), isoliquiritigenin (Yu et al., Brit. J. Pharmacol. 114 (1995), 1587) and various substituted pyrazole derivatives (WO 98/16223).

In addition, WO 98/16507, WO 98/23619, WO 00/06567, WO 00/06568, WO 00/06569 and WO 00/21954 describe pyrazolopyridine derivatives as stimulators of soluble guanylate cyclase.

It has now been found, surprisingly, that the effect of the direct soluble guanylate cyclase stimulators described above can be enhanced on administration of a lipid-lowering agent in combination with these soluble guanylate cyclase stimulators.

The present invention further relates to the use of lipid-lowering agents for enhancing the effect of direct soluble guanylate cyclase stimulators in the treatment of diseases which can be influenced by stimulation of soluble guanylate cyclase.

It is possible in this way for example to reduce the amount of direct soluble guanylate cyclase stimulator, or amount of lipid-lowering agent, which are necessary for the treatment of diseases and thus diminish the potential for side effects.

The present invention thus relates to a combination product comprising

as active ingredient component A at least one direct soluble guanylate cyclase stimulator; and

as active ingredient component B at least one lipid-lowering agent.

The term “combination product” as used for the purposes of the present invention means that the two active ingredient components A and B can be administered either simultaneously or sequentially (i.e. separately from one another).

The term “combination product” thus encompasses, according to the invention, ingredients A and B either in one functional unit, i.e. as true combination (e.g. as mixture, mix or blend), or else (spatially) separate in juxtaposition, i.e. as so-called kit of parts.

The present invention thus further relates also to a combination therapy for diseases which can be influenced by stimulation of soluble guanylate cyclase using a combination product which comprises at least one direct soluble guanylate cyclase stimulator and at least one lipid-lowering agent.

As mentioned previously, the combination of the invention can be administered, i.e. the combination therapy of the invention can take place, in such a way that the active ingredient components A and B are administered simultaneously. It is possible in this case for the active ingredient components A and B, as described above, to be present either in one functional unit (i.e. as true combination such as, for example, as mixture, mix or blend) or else (spatially) separate in juxtaposition (i.e. as so-called kit or kit-of-parts).

In a preferred embodiment of the present invention, the active ingredient components A and B are administered separately from one another, in particular sequentially.

This can take place for example by administering a daily dose of the lipid-lowering agent some days (e.g. about 1 week or else only 1-4 days) before administration of the direct soluble guanylate cyclase stimulator.

It is also possible to administer the direct soluble guanylate cyclase stimulator within a pre-existing lipid-lowering agent therapy, for example for patients with severe hypercholesterolemia, in whom the elevated cholesterol levels are already treated permanently with lipid-lowering agents. In this case, therefore, administration of the lipid-lowering agent can also be continued before and in parallel with the administration of the direct soluble guanylate cyclase stimulator.

In a preferred embodiment of the present invention, the active ingredient components A and B of the combination product of the invention are thus administered sequentially, preferably the lipid-lowering agent preceding, i.e. prior to, administration of the direct soluble guanylate cyclase stimulator.

Without wishing in this connection to be bound to a particular theory, the improvement in the effect of the direct soluble guanylate cyclase stimulator through simultaneous, sequential or parallel administration of lipid-lowering agents can presumably be explained by the fact that the lipid-lowering agents improve the impaired endothelial function by generating nitric oxide (NO) (Current Opinion in Lipidology, 1997, Vol. 8, pages 362-368 and Circulation 1998, 97, pages 1129-1135). It has been possible to show that direct soluble guanylate cyclase stimulators show a synergistic effect in combination with NO (cf., for example, WO 00/06569, FIG. 1).

According to the present invention, the lipid-lowering agent can be selected from the group of:

HMG-CoA reductase inhibitors,

squalene synthase inhibitors,

bile acid absorption inhibitors (also called bile acid anion exchangers or bile acid sequestrants),

fibric acid and its derivatives,

nicotinic acid and its analogs and

ω3-fatty acids.

For further details of the aforementioned lipid-lowering agents, reference is made in this connection to the article by Gilbert R. Thompson & Rissitaza P. Naoumova “New prospects for lipid-lowering drugs” in Exp. Opin. Invest. Drugs (1998), 7(5), pages 715-727, the entire contents of which are hereby expressly incorporated by reference.

The lipid-lowering agents preferred according to the invention amongst those aforementioned are the HMG-CoA reductase inhibitors. The abbreviation “HMG-CoA” in this connection stands for “3-hydroxymethylglutaryl-coenzyme A”.

In turn, the HMG-CoA reductase inhibitors particularly preferred according to the invention belong to the substance class of vastatins—usually referred to only as “statins” for simplicity in the literature.

Those statins which are in turn particularly preferred according to the invention are

atorvastatin (commercially available under the name Lipitor® from Parke-Davis);

cerivastatin (commercially available under the name Lipobay® or Baycol® from Bayer);

fluvastatin (commercially available under the name Lescol® from Novartis);

lovastatin (commercially available under the name Mevacor® from Merck);

pravastatin (commercially available under the name Lipostat® from Bristol-Myers Squibb);

simvastatin (commercially available under the name Zocor® from Merck);

pitavastatin (also called “nisvastatin”; NK-104; systematic name: [S-[R*,S*(E)-]]-7-[2-cyclopropyl-4-(4-fluorophenyl)-3-quinolinyl]-3,5-dihydroxy-6-heptenoic acid);

dalvastatin;

mevastatin;

dihydrocompactin;

compactin; and

rosuvastatin (commercially available under the name Crestor® from AstraZeneca; systematic name: (+)-(3R,5S)bis(7-(4-(4-fluorophenyl)-6-isopropyl-2-(N-methyl-N-methanesulfonylamino)pyrimidin-5-yl)-3,5-dihydroxy-6(E)-heptenoic acid);

and the respective salts, hydrates, alcoholates, esters and tautomers thereof.

Very particularly preferred among these are atorvastatin, cerivastatin, fluvastatin, lovastatin, pravastatin, pitavastatin, simvastatin and rosuvastatin, and the respective salts, hydrates, alcoholates, esters and tautomers thereof.

Very particularly preferred among these in turn are cerivastatin and atorvastatin and the respective salts, hydrates, alcoholates, esters and tautomers thereof.

For further details of the aforementioned statins, reference is made to the discussiones in Drugs of the Future 1994, 19(6), pages 537-541 and 1995, 20(6), page 611 and 1996, 21(6), page 642, the contents of each of which are incorporated in their entirety by reference.

The term “salt” for the purposes of the present invention means in each case physiologically acceptable salts of the respective compounds. These may be, for example: salts of mineral acids, carboxylic acids or sulfonic acids, in particular with hydrochloric acid, hydrobromic acid or sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, fumaric acid, maleic acid or benzoic acid or of mixed salts thereof. However, salts with conventional bases are also possible, such as, for example, alkali metal salts (e.g. sodium or potassium salts), alkaline earth metal salts (e.g. calcium or magnesium salts) or ammonium salts derived from ammonia or organic amines such as, for example, diethylamine, triethylamine, ethyldiisopropylamine, procain, dibenzylamine, N-methylmorpholine, dihydroabietylamine, 1-ephenamine or methylpiperidine and mixed salts thereof.

Examples of statin salts which can be used according to the invention are fluindostatin (the monosodium salt of fluvastatin); the monopotassium salt and the calcium salt of pitavastatin; and the calcium salt of (+)-(3R,5S)bis(7-7-(4-(4-fluorophenyl)-6-isopropyl-2-(N-methyl-N-methanesulfonylamino)pyrimidin-5-yl)-3,5-dihydroxy-6(E)-heptenoic acid (“rosuvastatin”, “ZD 4522” or “S 4522” from Shionogi or AstraZeneca). Further examples of statin salts which can be used according to the invention are the monosodium and monopotassium salts, and the calcium salts of cerivastatin, of atorvastatin and of pravastatin.

Further preferred HMG-CoA reductase inhibitors are described in EP-A-0 325 130 and in EP-A-O-491 226, the contents of which are hereby incorporated by reference. EP-A-0 325 130 relates to substituted pyridines, and EP-A-O-491 226 describes substituted pyridyldihydroxyheptenoic acid derivatives and their salts, particularly including cerivastatin which is particularly preferred according to the invention (claim 6 of EP-A-O-491 226).

Likewise preferred according to the invention are the statins mentioned in WO-A-99/11263, the disclosure of which is incorporated by reference.

Equally preferred according to the invention are the HMG-CoA reductase inhibitors mentioned in the publication Bioorganic & Medicinal Chemistry, Vol. 5, No. 2, pages 437-444 (1997), the disclosure of which is hereby incorporated in its entirety by reference.

A further review of HMG-CoA reductase inhibitors is present in Pharmazie in unserer Zeit, Vol. 28, No. 3, pages 147-1152 (1999).

The aforementioned bile acid absorption inhibitors (bile acid sequestrants) which are preferred according to the invention are cholestyramine (commercially availlable under the name Qestran® from Bristol-Myers Squibb) and colestipol (commercially available under the name Colestid® from Pharmacia & Upjohn) (see also Exp. Opin. Invest. Drugs (1998), 7(5), pages 715-727).

The aforementioned fibrinc acid derivatives which are preferred according to the invention are ciprofibrate (commercially available under the name Modalim® from Sanofi Winthrop), fenofibrate (commercially available under the name Lipantil® from Fournier), gemfibrozil (commercially available under the name Lopid® from Parke-Davis), bezafibrate and clofibrate (see also Exp. Opin. Invest. Drugs (1998), 7(5), pages 715-727).

Of the aforementioned nicotinic acid analogs, preference is given according to the invention to acipimox (commercially available under the name Olbetam® from Pharmacia & Upjohn) (see also Exp. Opin. Invest. Drugs (1998), 7(5), pages 715-727).

Of the aforementioned co3-fatty acids, preference is given according to the invention to maxepa (marketed by Seven Seas) (in this connection, see also Exp. Opin. Invest. Drugs (1998), 7(5), pages 715-727).

According to the present invention, the direct soluble guanylate cyclase stimulator can preferably be selected from the compounds described in the publication WO WO 00/06569 and WO 00/21954. The content of these publications is expressly incorporated by reference.

It is particularly preferred to use a direct soluble guanylate cyclase stimulator of the following formula (I):

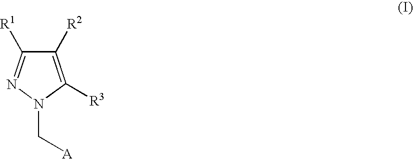

in which

R^{I} is a saturated, partly unsaturated or aromatic 5- or 6-membered heterocycle having up to 3 heteroatoms from the series S, N and/or O, which may be bonded via a nitrogen atom and which is optionally substituted up to 3 times, identically or differently, by

amino, azido, formyl, mercaptyl, carboxyl, hydroxyl, straight-chain or branched and optionally halogen-, acyloxy-, arylthio- or heteroarylthio-substituted acyl, alkoxy, alkylthio or alkoxycarbonyl each having up to 6 carbon atoms, nitro, cyano, halogen, C₆₋₁₀-aryl which is optionally substituted by straight-chain or branched alkyl having up to 4 carbon atoms, or NO, NHCO—C₁₋₆-alkyl, N(CO—C₁₋₆-alkyl)₂, NHSO₂—C₁₋₆-alkyl;

a radical of the formula R^{I}NCOR^{II} which is bonded via the nitrogen atom to the remainder of the molecule, where

R^{I} and R^{II} together with the amide group to which they are bonded form a five- to seven-membered heterocycle which may be saturated or partly unsaturated, may optionally contain a further heteroatom from the group of N, O, S, and may have one to five further substituents from the group of oxo, C₁₋₆-alkyl, hydroxyl, hydroxy-C₁₋₆-alkyl, halogen, or may be fused to a C₆₋₁₀-aryl ring or to a C₃₋₈-cycloalkyl ring in which two carbon atoms are optionally linked together via an oxygen atom;

a radical from the group consisting of —OSO₂—C₁₋₆-alkyl, —OSO₂—C₃₋₈-cycloalkyl, —OSO₂-phenyl, where the phenyl ring may optionally be substituted;

a radical of the formula —O—CX—NR^{III}R^{IV}, where

X is O or S;

R^{III} and R^{IV} may be identical or different from one another and is a radical from the group consisting of H, optionally substituted C₁₋₆-alkyl, optionally substituted C₁₋₆-alkoxy-C₁₋₆-alkyl, optionally substituted hydroxy-C₁₋₆-alkyl, optionally substituted C₂₋₆-alkenyl, optionally substituted C₁₋₆-alkylcarbonyloxy-C₁₋₆-alkyl, optionally substituted hydroxycarbonyl-C₁₋₆-alkyl, phenyl which is optionally substituted by a C₁₋₆-alkyl radical, or a saturated five- to seven-membered heterocycle which is optionally linked via a C₁₋₆-alkyl radical to the nitrogen atom, or optionally substituted C₃₋₈-cycloalkyl, where R³ and R⁴ cannot both be H; or

R^{III} and R^{IV} together with the nitrogen atom to which they are bonded form a five- to seven-membered saturated heterocycle which may optionally contain a further heteroatom from the group of N, O, S and/or optionally be substituted or fused to a phenyl ring;

a radical of the formula NR^{V}SO₂R^{VI} in which

R^{V} and R^{VI} together with the heteroatoms to which they are bonded form a five- to seven-membered heterocycle which may be saturated or partially unsaturated, may optionally comprise one or more further heteroatoms from the group of N, O, S, and may optionally be substituted;

straight-chain or branched alkenyl or alkynyl having up to 10 carbon atoms or straight-chain or branched alkyl having up to 20 carbon atoms, which may itself optionally be substituted by hydroxyl, amino, azido, carboxyl, straight-chain or branched acyl, alkoxy, alkoxycarbonyl or acylamino having up to 5 carbon atoms, aryl having 6 to 10 carbon atoms, a 5- to 6-membered aromatic heterocycle having up to 0.3 heteroatoms from the series S, N and/or O, halogen, cyano, dialkylamino having up to 6 carbon atoms, alkylamino having up to 6 carbon atoms and/or cycloalkyl having 3 to 8 carbon atoms or by a radical of the formula —OR⁴ in which R⁴ is straight-chain or branched acyl having up to 5 carbon atoms,

saturated or partially unsaturated C₃-C₈-cycloalkyl which may optionally be substituted one or more times by amino, azido, formyl, mercaptyl, carboxyl, hydroxyl, morpholino, piperidino, pyrrolidino, sulfonamino, straight-chain, cyclic or branched acyl, acylamino, alkoxy, benzyloxy, alkylamino, dialkylamino, alkylsulfonyl, alkylsulfonamino, alkylthio, alkoxycarbonyl each having up to 6 carbon atoms, nitro, cyano, halogen, phenyl and/or is optionally substituted by straight-chain or branched or cyclic alkyl having up to 6 carbon atoms which may in turn be substituted by amino, mercaptyl, carboxyl, hydroxyl, morpholino, piperidino, pyrrolidino, straight-chain, cyclic or branched acyl, acylamino, alkoxy, alkylamino, dialkylamino, alkylsulfonyl, alkylthio, phenyl, alkylsulfonamino, alkoxycarbonyl each having up to 6 carbon atoms, nitro, cyano, halogen,

a 3-8-membered ring which may be saturated, unsaturated or partially unsaturated, comprises 1-4 heteroatoms from the series N, O, S, SO, SO₂ and which may also be linked via N, with particular preference for imidazolyl, imidazolinyl, imidazolidinyl, morpholino, piperidine, piperazine, pyrrolidine, triazolyl, pyrrole, pyridine, thiomorpholino, s-oxothiomorpholino and S,S-dioxothiomorpholino or a radical of the formula

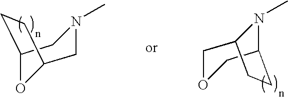

in which n is 1 or 2;

and which is optionally substituted one or more times by a 5- or 6-membered ring which comprises two oxygen atoms as ring members and forms with the 3-8-membered ring a bicyclic unit or a spiro unit, and/or hydroxyl, cyano, straight-chain or branched alkyl, acyl or alkoxycarbonyl each having up to 6 carbon atoms, where alkyl, acyl and alkoxycarbonyl may be substituted by hydroxyl, amino, halogen, carboxyl, straight-chain or branched acyl, alkoxy, alkoxycarbonyl or acylamino each having up to 5 carbon atoms,

a radical of the formula

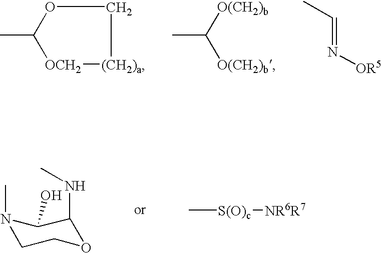

in which

a, b and b′ are identical or different and are a number 0, 1, 2 or 3,

R⁵ is hydrogen or straight-chain or branched alkyl having up to 4 carbon atoms,

c is a number 1 or 2, and

R⁶ and R⁷ are identical or different and are hydrogen or straight-chain or branched alkyl having up to 10 carbon atoms, which is optionally substituted by cycloalkyl having 3 to 8 carbon atoms or by aryl having 6 to 10 carbon atoms, which may in turn be substituted by halogen, or aryl having 6 to 10 carbon atoms which is optionally substituted by halogen, or cycloalkyl having 3 to 7 carbon atoms, or R⁶ and R⁷ together with the nitrogen atom form a 5-to 7-membered saturated heterocycle which may optionally comprise a further oxygen atom or a —NR⁸ radical, in which

R⁸ is hydrogen, straight-chain or branched alkyl having up to 4 carbon atoms or a radical of the formula

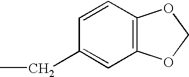

or benzyl or phenyl, where the ring systems are optionally substituted by halogen,

radicals of the formulae —SO₃H or —S(O)_{d}R⁹ in which

d is a number 1 or 2,

R⁹ is straight-chain or branched alkyl having 1 to 10 carbon atoms, cycloalkyl having 3 to 8 carbon atoms, aryl having 6 to 10 carbon atoms or a saturated or unsaturated 5- to 6-membered heterocycle having up to 3 heteroatoms from the series S, N and/or O, where the ring systems may optionally be substituted by halogen or by straight-chain or branched alkyl or alkoxy each having up to 4 carbon atoms,

a radical of the formula PO(OR¹⁰)(OR¹¹) in which

R¹⁰ and R¹¹ are identical or different and are hydrogen, straight-chain or branched alkyl having up to 8 carbon atoms or cycloalkyl having 3 to 8 carbon atoms, aryl having 6 to 10 carbon atoms or benzyl,

oxycycloalkyl having 3 to 8 ring members or radicals of the formulae —CON═C(NH₂)₂, —C═NH(NH₂), —NH—C(═NH)NH₂ or (CO)ₑNR¹²R¹³, in which

e is a number 0 or 1,

R¹² and R¹³ are identical or different and are hydrogen, straight-chain or branched alkyl having up to 14 carbon atoms or cycloalkyl having 3 to 14 carbon atoms, aryl having 6 to 10 carbon atoms or a saturated or unsaturated 3-to 10-membered ring having up to 5 heteroatoms from the series N, O, S, where said radicals may optionally be substituted by aryl having 6 to 10 carbon atoms, heterocyclyl, cycloalkyl having 3 to 7 carbon atoms, hydroxyl, amino or straight-chain or branched alkoxy, acyl or alkoxycarbonyl each having up to 6 carbon atoms,

and in the case where e is 1,

R¹² and R¹³ may also form, with inclusion of the nitrogen atom to which they are bonded, a 5- or 6-membered ring having up to 3 heteroatoms from the series N, O, S, which may optionally be substituted up to 3 times by hydroxyl, alkoxy or alkyl each having up to 8 carbon atoms,

and in the case where e is 0,

R¹² and R¹³ may also be straight-chain, branched or cyclic acyl having up to 14 carbon atoms, hydroxyalkyl, straight-chain or branched alkoxycarbonyl or acyloxyalkyl each having up to 6 carbon atoms or a radical of the formula —SO₂R¹⁴ in which

R¹⁴ is straight-chain or branched alkyl having up to 4 carbon atoms,

with the proviso that in the case where e is 0, R¹² and R¹³ are not both hydrogen,

a purine residue which may optionally be substituted up to three times by halogen, azido, cyano, hydroxyl, amino, monoalkylamino having up to 5 carbon atoms, dialkylamino having in each case up to 5 carbon atoms, alkyl having up to 5 carbon atoms and/or alkoxy having up to 5 carbon atoms,

R² and R³ form, with inclusion of the double bond, a 6-membered saturated or aromatic heterocycle having up to 3 heteroatoms from the series N, S and/or O or a phenyl ring which are optionally substituted up to 3 times identically or differently by formyl, mercaptyl, carboxyl, hydroxyl, amino, straight-chain or branched acyl, alkylthio, alkoxy, alkoxycarbonyl having in each case up to 6 carbon atoms, nitro, cyano, azido, halogen, phenyl or straight-chain or branched alkyl having up to 6 carbon atoms which may in turn be substituted by hydroxyl, amino, carboxyl, straight-chain or branched acyl, alkoxy or alkoxycarbonyl each having up to 5 carbon atoms,

A is phenyl or a 5- to 6-membered aromatic or saturated heterocycle having up to 3 heteroatoms from the series S, N and/oder O, each of which is optionally substituted up to 3 times identically or differently by mercaptyl, hydroxyl, formyl, carboxyl, straight-chain or branched acyl, alkylthio, alkyloxyacyl, alkoxy or alkoxycarbonyl each having up to 6 carbon atoms, nitro, cyano, trifluoromethyl, azido, halogen, phenyl or straight-chain or branched alkyl having up to 6 carbon atoms which may in turn be substituted by hydroxyl, carboxyl, straight-chain or branched acyl, alkoxy or alkoxycarbonyl each having up to 5 carbon atoms, and/or are substituted by a group of the formula —(CO)_{f}—NR¹⁵R¹⁶ in which

d is a number 0 or 1,

R¹⁵ and R¹⁶ are identical or different and are hydrogen, phenyl, benzyl or straight-chain or branched alkyl or acyl each having up to 5 carbon atoms,

and the isomeric forms and salts thereof and the N-oxides thereof.

It is particularly preferred according to the invention to use a direct soluble guanylate cyclase stimulator of the formula (Ia)

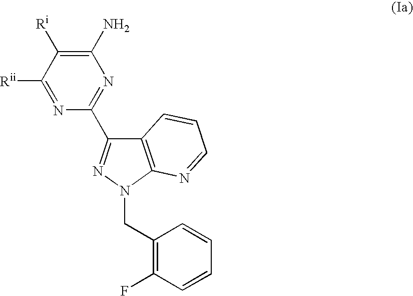

in which

Rⁱ is a saturated or unsaturated, optionally substituted C₃₋₈-cycloalkyl, or

is a saturated, unsaturated or partially unsaturated 3-8-membered heterocycle which may comprise 1-4 heteroatoms from the series N, O, S, SO, SO₂ and optionally be substituted, or

is a radical of the formula RⁱⁱⁱNCORⁱⁱⁱⁱ which is bonded via the nitrogen atom to the remainder of the molecule, where Rⁱⁱⁱ and Rⁱⁱⁱ together with the amide group to which they are bonded form a five- or six-membered saturated heterocycle which may optionally comprise a further oxygen atom and may have one to five further substituents from the group of oxo, C₁₋₄-alkyl, or may be fused with a phenyl ring; or

is 4-pyridinyl or 3-pyridinyl;

Rⁱⁱ is H, halogen or NH₂, or

Rⁱ and Rⁱⁱ together form a radical of the formula

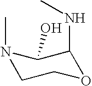

It is preferred according to the present invention to use compounds of the formula (Ia) in which

Rⁱ is an optionally substituted cyclopropyl, cyclobutyl, cyclopentenyl, cyclopentyl, cyclohexyl, 1-hydroxycyclopropyl or 1-(fluoromethyl)cyclopropyl radical or is optionally substituted morpholino, piperidine, piperazine, pyrrolidine, 4-pyridinyl or 3-pyridinyl, triazolyl or thiomorpholino;

Rⁱⁱ is H, halogen or NH₂, or

Rⁱ and Rⁱⁱ together form a radical of the formula

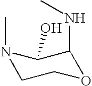

It is preferred according to the present invention to use a compound of the formula (Ia) in which

Rⁱ is a cyclopropyl radical, a 1-hydroxyl-cyclopropyl radical, morpholino, 4-pyridinyl or 3-pyridinyl,

Rⁱⁱ is H or NH₂, or

Rⁱ and Rⁱⁱ together form a radical of the formula

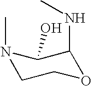

The compounds of the invention of the general formula (I) may also exist in the form of their salts. In general, mention may be made here of salts with organic or inorganic bases or acids.

Physiologically acceptable salts are preferred for the purposes of the present invention. Physiologically acceptable salts of the compounds of the invention may be salts of the substances of the invention with mineral acids, carboxylic acids or sulfonic acids. Particularly preferred examples are salts with hydrochloric acid, hydrobromic acid, sulfuric acid, phosphoric acid, methanesulfonic acid, ethanesulfonic acid, p-toluenesulfonic acid, benzenesulfonic acid, naphthalenedisulfonic acid, acetic acid, propionic acid, lactic acid, tartaric acid, citric acid, fumaric acid, maleic acid or benzoic acid.

Physiologically acceptable salts may be metal or ammonium salts of the compounds of the invention having a free carboxyl group. Particularly preferred examples are sodium, potassium, magnesium or calcium salts, and ammonium salts derived from ammonia or organic amines such as, for example, ethylamine, di- or triethylamine, di- or triethanolamine, dicyclohexylamine, dimethylaminoethanol, arginine, lysine or ethylenediamine.

The compounds of the invention may exist in stereoisomeric forms which either are related as image and mirror image (enantiomers) or which are not related as image and mirror image (diastereomers). The invention relates both to the enantiomers or diastereomers and to respective mixtures thereof. The racemic forms can, just like the diastereomers, be separated in a known manner, for example by chromatographic separation, into the stereoisomerically pure constituents. Double bonds present in the compounds of the invention may be in the cis or trans configuration (Z or E form).

In addition, certain compounds may exist in tautomeric forms. This is known to the skilled worker, and such compounds are likewise encompassed by the invention.

The compounds of the invention may additionally occur in the form of their hydrates, with the number of water molecules bound to the molecule depending on the particular compound of the invention.

Unless indicated otherwise, the substituents have for the purposes of the present invention in general the following meanings:

Alkyl is generally a straight-chain or branched hydrocarbon radical having 1- to 20 carbon atoms. Examples which may be mentioned are methyl, ethyl, propyl, isopropyl, butyl, isobutyl, pentyl, isopentyl, hexyl, isohexyl, heptyl, isoheptyl, octyl and isooctyl, nonyl, decyl, dodeyl, eicosyl.

Alkenyl is generally a straight-chain or branched hydrocarbon radical having 2 to 20 carbon atoms and one or more, preferably having one or two, double bonds. Examples which may be mentioned are allyl, propenyl, isopropenyl, butenyl, isobutenyl, pentenyl, isopentenyl, hexenyl, isohexenyl, heptenyl, isoheptenyl, octenyl, isooctenyl.

Alkynyl is generally a straight-chain or branched hydrocarbon radical having 2 to 20 carbon atoms and one or more, preferably having one or two, triple bonds. Examples which may be named are ethynyl, 2-butynyl, 2-pentinyl and 2-hexynyl.

Acyl is generally straight-chain or branched lower alkyl having 1 to 9 carbon atoms which is linked via a carbonyl group. Examples which may be mentioned are: acetyl, ethylcarbonyl, propylcarbonyl, isopropylcarbonyl, butylcarbonyl and isobutylcarbonyl.

Alkoxy is generally a straight-chain or branched hydrocarbon radical having 1 to 14 carbon atoms which is linked via an oxygen atom. Examples which may be mentioned are methoxy, ethoxy, propoxy, isopropoxy, butoxy, isobutoxy, pentoxy isopentoxy, hexoxy, isohexoxy, heptoxy, isoheptoxy, octoxy or isooctoxy. The terms “alkoxy” and “alkyloxy” are used synonymously.

Alkoxyalkyl is generally an alkyl radical having up to 8 carbon atoms which is substituted by an alkoxy radical having up to 8 carbon atoms.

Alkoxycarbonyl can be represented for example by the formula

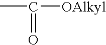

Alkyl in this case is generally a straight-chain or branched hydrocarbon radical having 1 to 13 carbon atoms. The following alkoxycarbonyl radicals may be mentioned for example: methoxycarbonyl, ethoxycarbonyl, propoxycarbonyl, isopropoxycarbonyl, butoxycarbonyl or isobutoxycarbonyl.

Cycloalkyl is generally a cyclic hydrocarbon radical having 3 to 8 carbon atoms. Cyclopropyl, cyclopentyl and cyclohexyl are preferred. Examples which may be mentioned are cyclopentyl, cyclohexyl, cycloheptyl and cyclooctyl.

Cycloalkoxy is for the purposes of the invention an alkoxy radical whose hydrocarbon radical is a cycloalkyl radical. The cycloalkyl radical generally has up to 8 carbon atoms. Examples which may be mentioned are: cyclopropyloxy and cyclohexyloxy. The terms “cycloalkoxy” and “cycloalkyloxy” are used synonymously.

Aryl is generally an aromatic radical having 6 to 10 carbon atoms. Phenyl and naphthyl are preferred aryl radicals.

Halogen is for the purposes of the invention fluorine, chlorine, bromine and iodine.

Heterocycle is for the purposes of the invention generally a saturated, unsaturated or aromatic 3- to 10-membered, for example 5- or 6-membered, heterocycle which may comprise up to 3 heteroatoms from the series S, N and/or O, and which in the case of a nitrogen atom may also be bonded via the latter. Examples which may be mentioned are: oxadiazolyl, thiadiazolyl, pyrazolyl, pyridyl, pyrimidinyl, pyridazinyl, pyrazinyl, thienyl, furyl, pyrrolyl, pyrrolidinyl, piperazinyl, tetrahydropyranyl, tetrahydrofuranyl, 1,2,3 triazolyl, thiazolyl, oxazolyl, imidazolyl, morpholinyl or piperidyl. Thiazolyl, furyl, oxazolyl, pyrazolyl, triazolyl, pyridyl, pyrimidinyl, pyridazinyl and tetrahydropyranyl are preferred. The term “heteroaryl” (or “hetaryl”) stands for an aromatic heterocyclic radical.

Apart from the two active ingredient components A and B mentioned above, the combination product of the invention may also comprise any other active ingredients as long as they do not conflict with the area of indications and do not impair the effect of the direct soluble guanylate cyclase stimulator and of the lipid-lowering agent. In particular, it is possible to add to the composition of the invention organic nitrates or NO donors—that is to say compounds which stimulate the synthesis of cGMP—or compounds which inhibit the breakdown of cyclic guanosine monophosphate (cGMP).

Organic nitrates and NO donors for the purposes of the invention are generally substances which display their therapeutic effect via release of NO or NO species. Sodium nitroprusside, nitroglycerine, isosorbide dinitrate, isosorbide mononitrate, molsidomine and SIN-1 are preferred.

The invention additionally encompasses combination with compounds which inhibit the breakdown of cyclic guanosine monophosphate (cGMP). These are, in particular, inhibitors of phosphodiesterases 1, 2′ and 5; nomenclature of Beavo and Reifsnyder (1990) TiPS 11 pages 150 to 155. These inhibitors potentiate the effect of the compound of the invention and increase the desired pharmacological effect.

These other active ingredients which are preferably present may—just like the active ingredient components A and B—be present either as true mixture together with A and/or B or else be present spatially separate therefrom. Administration thereof can take place in parallel or simultaneously or sequentially in relation to the active ingredient component(s) A and/or B.

The other active ingredients preferably present in the combination product of the invention include, for example:

other active ingredients improving erectile ability, for example: cGMP PDE inhibitors such as, for example, sildenafil (EP-B-0 463 756), IC 351 (WO 95/19978) or vardenafil (WO 99/24433), α-adrenergic antagonists such as, for example, yohimbin or Vasomax® from Zonagen; or else substances like those mentioned in WO-A-98/52569, the contents of which are hereby included by reference; or prostaglandins E1; or seretonin antagonists;

active ingredients from the cardiovascular area of indications;

active ingredients from the CNS and cerebral areas of indications;

vitamins;

minerals;

trace elements.

All conventional administration forms are suitable in each case for administering the two active ingredient components A and B (and the other active ingredients present where appropriate). Administration preferably takes place orally, perlingually, sublingually, nasally, transdermally, buccally, intravenously, rectally, by inhalation or parenterally. Administration preferably takes place orally, sublingually or nasally. Oral administration is very particularly preferred.

It is additionally possible to administer the two active ingredient components A and B in different dosage forms if administration is spatially separate or at different times.

The two active ingredient components A and B can be converted—together or spatially separate—in each case in a manner known per se into the conventional formulations such as tablets, coated tablets, pills, granules, aerosols, syrups, emulsions, suspensions and solutions, using inert, nontoxic, pharmaceutically suitable carriers or solvents. In these cases, the therapeutically active components A and B should each be present in a concentration of about 0.5 to 90% by weight of the complete mixture, i.e. in amounts which suffice to reach the stated dosage range.

The formulations are produced for example by extending the two active ingredient components A and B with solvents and/or carriers, where appropriate using emulsifiers and/or dispersants, it being possible, for example in the case where water is used as diluent, where appropriate to use organic solvents and auxiliary solvents.

The dosages administered on oral administration for human use are from 0.001 to 50 mg/kg, preferably from 0.001 mg/kg to 20 mg/kg, in particular 0.001 to 10 mg/kg, of body weight, particularly preferably 0.001 mg/kg to 5 mg/kg, of the respective active ingredient component A or B, to achieve effective and worthwhile results.

It may nevertheless be necessary where appropriate to depart from the amounts mentioned here, in particular depending on the body weight and the nature of the administration route, or on the individual behavior toward the combination product, on the nature of the formulation and on the time or interval over which administration takes place. Thus, it may be sufficient in some cases to make do with less than the aforementioned minimum amount, while in other cases the upper limit mentioned must be exceeded.

It may be advisable in the case where relatively large amounts are administered for these to be distributed in a plurality of single doses over the day.

1. A combination product comprising as pharmaceutically active ingredients at least one active ingredient component A and at least one active ingredient component B, characterized in that the active ingredient component A is a direct soluble guanylate cyclase stimulator and the active ingredient component B is a lipid-lowering agent.

2. The combination product as claimed in claim 1 for the treatment of diseases.

3. The combination product as claimed in one of claims 1 or 2, characterized in that the two active ingredient components A and B are administered separately from one another, in particular sequentially.

4. The combination product as claimed in any of claims 1 to 3, characterized in that the active ingredient components A and B are present as functional unit, in particular in the form of a mixture, of a mix or of a blend.

5. The combination product as claimed in any of claims 1 to 4, characterized in that active ingredient components A and B are present (spatially) separate from one another, in particular as kit-of-parts.

6. The combination product as claimed in any of claims 1 to 5, characterized in that the lipid-lowering agent (active ingredient component B) is selected from the group of (a) HMG-CoA reductase inhibitors; (b) squalene synthase inhibitors; (c) bile acid absorption inhibitors (,,bile acid sequestrants”); (d) fibric acid and its derivatives; (e) nicotinic acid and its analogs; (f) ω3-fatty acids.

7. The combination product as claimed in claim 6, characterized in that the lipid-lowering agent (active ingredient component B) is a HMG-CoA reductase inhibitor and is selected in particular from the group of statins, preferably from the group of atorvastatin, cerivastatin, fluvastatin, lovastatin, pravastatin, pitavastatin, simvastatin and rosuvastatin, and the respective salts, hydrates, alcoholates, esters and tautomers thereof.

8. The combination product as claimed in claim 7, characterized in that the lipid-lowering agent (active ingredient component B) is atorvastatin or its salt, hydrate, alcoholate, ester and tautomer.

9. The combination product as claimed in claim 7, characterized in that the lipid-lowering agent (active ingredient component B) is cerivastatin or its salt, hydrate, alcoholate, ester and tautomer.

10. The combination product as claimed in any of claims 1 to 9, characterized in that the direct soluble guanylate cyclase stimulator (active ingredient component A) is selected from compounds of the following formula (I): 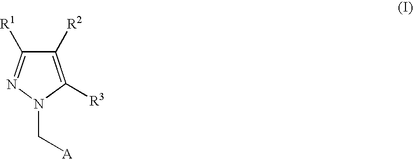
in which
R^{I} is a saturated, partly unsaturated or aromatic 5- or 6-membered heterocycle having up to 3 heteroatoms from the series S, N and/or O, which may be bonded via a nitrogen atom and which is optionally substituted up to 3 times, identically or differently, by
amino, azido, formyl, mercaptyl, carboxyl, hydroxyl, straight-chain or branched and optionally halogen-, acyloxy-, arylthio- or heteroarylthio-substituted acyl, alkoxy, alkylthio or alkoxycarbonyl each having up to 6 carbon atoms, nitro, cyano, halogen, C₆₋₁₀-aryl which is optionally substituted by straight-chain or branched alkyl having up to 4 carbon atoms, or NO, NHCO—C₁₋₆-alkyl, N(CO—C₁₋₆-alkyl)₂, NHSO₂—C₁₋₆-alkyl;
a radical of the formula R^{I}NCOR^{II} which is bonded via the nitrogen atom to the remainder of the molecule, where
R^{I} and R^{II} together with the amide group to which they are bonded form a five- to seven-membered heterocycle which may be saturated or partly unsaturated, may optionally contain a further heteroatom from the group of N, O, S, and may have one to five further substituents from the group of oxo, C₁₋₆-alkyl, hydroxyl, hydroxy-C₁₋₆-alkyl, halogen, or may be fused to a C₆₋₁₀-aryl ring or to a C₃₋₈-cycloalkyl ring in which two carbon atoms are optionally linked together via an oxygen atom;
a radical from the group consisting of —OSO₂—C₁₋₆-alkyl, —OSO₂—C₃₋₈-cycloalkyl, —OSO₂-phenyl, where the phenyl ring may optionally be substituted;
a radical of the formula —O—CX—NR^{III}R^{IV}, where
X is O or S;
R^{III} and R^{IV} may be identical or different from one another and is a radical from the group consisting of H, optionally substituted C₁₋₆-alkyl, optionally substituted C₁₋₆-alkoxy-C₁₋₆-alkyl, optionally substituted hydroxy-C₁₋₆-alkyl, optionally substituted C₂₋₆-alkenyl, optionally substituted C₁₋₆-alkylcarbonyloxy-C₁₋₆-alkyl, optionally substituted hydroxycarbonyl-C₁₋₆-alkyl, phenyl which is optionally substituted by a C₁₋₆-alkyl radical, or a saturated five- to seven-membered heterocycle which is optionally linked via a C₁₋₆-alkyl radical to the nitrogen atom, or optionally substituted C₃₋₈-cycloalkyl, where R³ and R⁴ cannot both be H; or
R^{III} and R^{IV} together with the nitrogen atom to which they are bonded form a five- to seven-membered saturated heterocycle which may optionally contain a further heteroatom from the group of N, O, S and/or optionally be substituted or fused to a phenyl ring;
a radical of the formula NR^{V}SO₂R^{VI} in which
R^{V} and R^{VI} together with the heteroatoms to which they are bonded form a five- to seven-membered heterocycle which may be saturated or partially unsaturated, may optionally comprise one or more further heteroatoms from the group of N, O, S, and may optionally be substituted;
straight-chain or branched alkenyl or alkynyl having up to 10 carbon atoms or straight-chain or branched alkyl having up to 20 carbon atoms, which may itself optionally be substituted by hydroxyl, amino, azido, carboxyl, straight-chain or branched acyl, alkoxy, alkoxycarbonyl or acylamino having up to 5 carbon atoms, aryl having 6 to 10 carbon atoms, a 5- to 6-membered aromatic heterocycle having up to 3 heteroatoms from the series S, N and/or O, halogen, cyano, dialkylamino having up to 6 carbon atoms, alkylamino having up to 6 carbon atoms and/or cycloalkyl having 3 to 8 carbon atoms or by a radical of the formula —OR⁴ in which R⁴ is straight-chain or branched acyl having up to 5 carbon atoms,
saturated or partially unsaturated C₃-C₈-cycloalkyl which may optionally be substituted one or more times by amino, azido, formyl, mercaptyl, carboxyl, hydroxyl, morpholino, piperidino, pyrrolidino, sulfonamino, straight-chain, cyclic or branched acyl, acylamino, alkoxy, benzyloxy, alkylamino, dialkylamino, alkylsulfonyl, alkylsulfonamino, alkylthio, alkoxycarbonyl each having up to 6 carbon atoms, nitro, cyano, halogen, phenyl and/or is optionally substituted by straight-chain or branched or cyclic alkyl having up to 6 carbon atoms which may in turn be substituted by amino, mercaptyl, carboxyl, hydroxyl, morpholino, piperidino, pyrrolidino, straight-chain, cyclic or branched acyl, acylamino, alkoxy, alkylamino, dialkylamino, alkylsulfonyl, alkylthio, phenyl, alkylsulfonamino, alkoxycarbonyl each having up to 6 carbon atoms, nitro, cyano, halogen,
a 3-8-membered ring which may be saturated, unsaturated or partially unsaturated, comprises 1-4 heteroatoms from the series N, O, S, SO, SO₂ and which may also be linked via N, with particular preference for imidazolyl, imidazolinyl, imidazolidinyl, morpholino, piperidine, piperazine, pyrrolidine, triazolyl, pyrrole, pyridine, thiomorpholino, S-oxothiomorpholino and S,S-dioxothiomorpholino or a radical of the formula 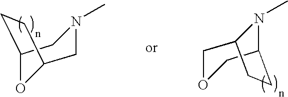
in which n is 1 or 2;
and which is optionally substituted one or more times by a 5- or 6-membered ring which comprises two oxygen atoms as ring members and forms with the 3-8-membered ring a bicyclic unit or a spiro unit, and/or hydroxyl, cyano, straight-chain or branched alkyl, acyl or alkoxycarbonyl each having up to 6 carbon atoms, where alkyl, acyl and alkoxycarbonyl may be substituted by hydroxyl, amino, halogen, carboxyl, straight-chain or branched acyl, alkoxy, alkoxycarbonyl or acylamino each having up to 5 carbon atoms,
a radical of the formula 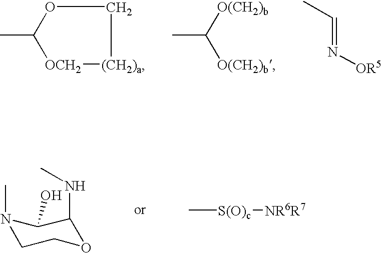
in which
a, b and b′ are identical or different and are a number 0, 1, 2 or 3,
R⁵ is hydrogen or straight-chain or branched alkyl having up to 4 carbon atoms,
c is a number 1 or 2, and
R⁶ and R⁷ are identical or different and are hydrogen or straight-chain or branched alkyl having up to 10 carbon atoms, which is optionally substituted by cycloalkyl having 3 to 8 carbon atoms or by aryl having 6 to 10 carbon atoms, which may in turn be substituted by halogen, or aryl having 6 to 10 carbon atoms which is optionally substituted by halogen, or cycloalkyl having 3 to 7 carbon atoms, or R⁶ and R⁷ together with the nitrogen atom form a 5-to 7-membered saturated heterocycle which may optionally comprise a further oxygen atom or a —NR⁸ radical, in which
R⁸ is hydrogen, straight-chain or branched alkyl having up to 4 carbon atoms or a radical of the formula 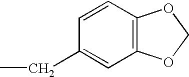
or benzyl or phenyl, where the ring systems are optionally substituted by halogen,
radicals of the formulae —SO₃H or —S(O)_{d}R⁹ in which
d is a number 1 or 2,
R⁹ is straight-chain or branched alkyl having 1 to 10 carbon atoms, cycloalkyl having 3 to 8 carbon atoms, aryl having 6 to 10 carbon atoms or a saturated or unsaturated 5- to 6-membered heterocycle having up to 3 heteroatoms from the series S, N and/or O, where the ring systems may optionally be substituted by halogen or by straight-chain or branched alkyl or alkoxy each having up to 4 carbon atoms,
a radical of the formula PO(OR¹⁰)(OR¹¹) in which
R¹⁰ and R¹¹ are identical or different and are hydrogen, straight-chain or branched alkyl having up to 8 carbon atoms or cycloalkyl having 3 to 8 carbon atoms, aryl having 6 to 10 carbon atoms or benzyl, oxycycloalkyl having 3 to 8 ring members or radicals of the formulae —CON═C(NH₂)₂, —C═NH(NH₂), —NH—C(═NH)NH₂ or (CO)ₑNR¹²R¹³, in which
e is a number 0 or 1,
R¹² and R¹³ are identical or different and are hydrogen, straight-chain or branched alkyl having up to 14 carbon atoms or cycloalkyl having 3 to 14 carbon atoms, aryl having 6 to 10 carbon atoms or a saturated or unsaturated 3-to 10-membered ring having up to 5 heteroatoms from the series N, O, S, where said radicals may optionally be substituted by aryl having 6 to 10 carbon atoms, heterocyclyl, cycloalkyl having 3 to 7 carbon atoms, hydroxyl, amino or straight-chain or branched alkoxy, acyl or alkoxycarbonyl each having up to 6 carbon atoms,
and in the case where e is 1,
R¹² and R¹³ may also form, with inclusion of the nitrogen atom to which they are bonded, a 5 or 6-membered ring having up to 3 heteroatoms from the series N, O, S, which may optionally be substituted up to 3 times by hydroxyl, alkoxy or alkyl each having up to 8 carbon atoms,
and in the case where e is 0,
R¹² and R¹³ may also be straight-chain, branched or cyclic acyl having up to 14 carbon atoms, hydroxyalkyl, straight-chain or branched alkoxycarbonyl or acyloxyalkyl each having up to 6 carbon atoms or a radical of the formula —SO₂R¹⁴ in which
R¹⁴ is straight-chain or branched alkyl having up to 4 carbon atoms,
with the proviso that in the case where e is 0, R¹² and R¹³ are not both hydrogen,
a purine residue which may optionally be substituted up to three times by halogen, azido, cyano, hydroxyl, amino, monoalkylamino having up to 5 carbon atoms, dialkylamino having in each case up to 5 carbon atoms, alkyl having up to 5 carbon atoms and/or alkoxy having up to 5 carbon atoms,
R² and R³ form, with inclusion of the double bond, a 6-membered saturated or aromatic heterocycle having up to 3 heteroatoms from the series N, S and/or O or a phenyl ring which are optionally substituted up to 3 times identically or differently by formyl, mercaptyl, carboxyl, hydroxyl, amino, straight-chain or branched acyl, alkylthio, alkoxy, alkoxycarbonyl having in each case up to 6 carbon atoms, nitro, cyano, azido, halogen, phenyl or straight-chain or branched alkyl having up to 6 carbon atoms which may in turn be substituted by hydroxyl, amino, carboxyl, straight-chain or branched acyl, alkoxy or alkoxycarbonyl each having up to 5 carbon atoms,
A is phenyl or a 5- to 6-membered aromatic or saturated heterocycle having up to 3 heteroatoms from the series S, N and/oder O, each of which is optionally substituted up to 3 times identically or differently by mercaptyl, hydroxyl, formyl, carboxyl, straight-chain or branched acyl, alkylthio, alkyloxyacyl, alkoxy or alkoxycarbonyl each having up to 6 carbon atoms, nitro, cyano, trifluoromethyl, azido, halogen, phenyl or straight-chain or branched alkyl having up to 6 carbon atoms which may in turn be substituted by hydroxyl, carboxyl, straight-chain or branched acyl, alkoxy or alkoxycarbonyl each having up to 5 carbon atoms, and/or are substituted by a group of the formula —(CO)_{f}—NR¹⁵R¹⁶ in which
d is a number 0 or 1,
R¹⁵ and R¹⁶ are identical or different and are hydrogen, phenyl, benzyl or straight-chain or branched alkyl or acyl each having up to 5 carbon atoms,
and the isomeric forms and salts thereof and the N-oxides thereof.

11. The combination product as claimed in claim 10, characterized in that the direct soluble guanylate cyclase stimulator is selected from compounds of the formula (Ia) 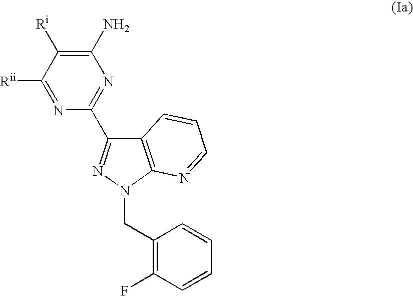
in which
Rⁱ is a saturated or unsaturated, optionally substituted C₃₋₈-cycloalkyl, or
is a saturated, unsaturated or partially unsaturated 3-8-membered heterocycle which may comprise 1-4 heteroatoms from the series N, O, S, SO, SO₂ and optionally be substituted, or
is a radical of the formula RⁱⁱⁱNCORⁱⁱⁱⁱ which is bonded via the nitrogen atom to the remainder of the molecule, where Rⁱⁱⁱ and Rⁱⁱⁱ together with the amide group to which they are bonded form a five- or six-membered saturated heterocycle which may optionally comprise a further oxygen atom and may have one to five further substituents from the group of oxo, C₁₋₄-alkyl, or may be fused with a phenyl ring; or
is 4-pyridinyl or 3-pyridinyl;
Rⁱⁱ is H, halogen or NH₂, or
Rⁱ and Rⁱⁱ together form a radical of the formula

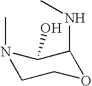

12. The combination product as claimed in claim 11, characterized in that
Rⁱ is an optionally substituted cyclopropyl, cyclobutyl, cyclopentenyl, cyclopentyl, cyclohexyl, 1-hydroxycyclopropyl or 1-(fluoromethyl)cyclopropyl radical or is optionally substituted morpholino, piperidine, piperazine, pyrrolidine, 4-pyridinyl or 3-pyridinyl, triazolyl or thiomorpholino;
Rⁱⁱ is H, halogen or NH₂, or
Rⁱ and Rⁱⁱ together form a radical of the formula

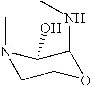

13. The combination product as claimed in claim 11, characterized in that
Rⁱ is a cyclopropyl radical, a 1-hydroxyl-cyclopropyl radical, morpholino, 4-pyridinyl or 3-pyridinyl,
Rⁱⁱ is H or NH₂, or
Rⁱ and Rⁱⁱ together form a radical of the formula

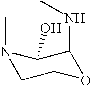

14. The use of lipid-lowering agents for increasing efficacy of direct soluble guanylate cyclase stimulators.

15. A process for producing compositions as claimed in any of claims 1 to 13, characterized in that at least one lipid-lowering agent and at least one direct soluble guanylate cyclase stimulator are converted, where appropriate with conventional excipients and additives, into a suitable administration form.

16. The use of compositions as claimed in any of claims 1 to 13 in the production of medicaments for the treatment of cardiovascular disorders.

17. The use of compositions as claimed in any of claims 1 to 13 in the production of medicaments for the treatment of hypertension.

18. The use of compositions as claimed in any of claims 1 to 13 in the production of medicaments for the treatment of thromboembolic disorders and ischaemias.

19. The use of compositions as claimed in any of claims 1 to 13 in the production of medicaments for the treatment of sexual dysfunction.

20. The use of compositions as claimed in any of claims 1 to 13 in the production of medicaments for the treatment of arteriosclerosis.

21. The use of compositions as claimed in any of claims 1 to 13 in the production of medicaments for the treatment of osteoporosis.

22. The use of compositions as claimed in any of claims 1 to 13 in the production of medicaments having an antiinflammatory effect.

23. The use of compositions as claimed in any of claims 1 to 13 in the production of medicaments for the treatment of disorders of the central nervous system.

24. The use as claimed in any of claims 16 to 23, where the compositions as claimed in any of claims 1 to 13 are employed in combination with organic nitrates or NO donors or in combination with compounds which inhibit the breakdown of cyclic guanosine monophosphate (cGMP).
